(19) United States
(12) Patent Application Publication
(10) Pub. No.: US 2012/0010360 A1
(43) Pub. Date: Jan. 12, 2012

(54) MODIFIED LAYERED MATERIAL AND UNSATURATED POLYESTER NANOCOMPOSITE COMPRISING THE SAME

(75) Inventors: Tsung-Yen Tsai, Jhongli City (TW); Chien-Hung Kuo, Jhongli City (TW); Cheng-Hsien Chung, Taipei County (TW); Chia-Hao Hsu, Taipei County (TW)

(73) Assignees: UNITED SHIP DESIGN & DEVELOPMENT CENTER, Taipei County (TW); CHUNG YUAN CHRISTIAN UNIVERSITY, Chung Li City (TW)

(21) Appl. No.: 13/241,282

(22) Filed: Sep. 23, 2011

Related U.S. Application Data
(62) Division of application No. 12/579,419, filed on Oct. 15, 2009.

(30) Foreign Application Priority Data
Jun. 3, 2009 (TW) 098118339

(51) Int. Cl. C08L 67/06; C07C 229/26; C08K 5/5455

(52) U.S. Cl. 524/724; 562/564

(57) ABSTRACT

A modified layered material is provided, which includes a layered inorganic material intercalated with an organic modifier. The organic modifier includes

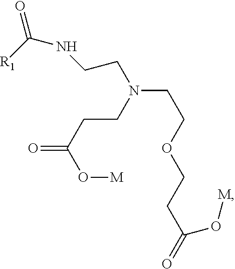

wherein M includes a metal and R₁ includes a carbon chain. An unsaturated polyester nanocomposite is also provided, which includes a polymer material including unsaturated polyester and the modified layered material, wherein the modified layered material is dispersed in the polymer material and is at least partially exfoliated.

### CROSS REFERENCE TO RELATED APPLICATIONS

This application is a Divisional of pending U.S. patent application Ser. No. 12/579,419, filed Oct. 15, 2009 and entitled “modified layered material and unsaturated polyester nanocomposite comprising the same”, which claims priority of Taiwan Patent Application No. 098118339, filed on Jun. 3, 2009, the entirety of which is incorporated by reference herein.

### BACKGROUND OF THE INVENTION

1. Field of the Invention

The present invention relates to modified layered material, and in particular relates to a modified layered material/unsaturated polyester nanocomposite comprising the same.

2. Description of the Related Art

Unsaturated polyester resins having unsaturated functional groups are suitable for the production of duroplastic articles by, for example crosslinking initiated by heating. Due to excellent characteristics including transparency, weatherability, and color fastness, unsaturated polyester resins are used in a wide variety of applications, such as construction materials, electronic materials, bathroom equipments, and plate materials.

During the molding process, unsaturated polyester resins shrink when curing, sometimes exceeding 10%. Accordingly, deformation or cracking may occur, thus resulting in uneven helical patterns (or so-called print-through phenomenon) being formed on the surface of the unsaturated polyester resins. For example, fiber reinforced plastic (FRP) plates composed of an unsaturated polyester resin and glass fibers often have uneven helical patterns formed on the surface thereof. Thus, shrinkage reducing agents or low-profile agents are added to unsaturated polyester resins to reduce shrinkage thereof. Commonly used shrinkage reducing agent includes polystyrene (PS), polymethylmethacrylate (PMMA), or/and styrene. However, often, the mechanical strength of the unsaturated polyester resin is decreased when shrinkage reducing agents are added, thus limiting applications thereof.

Thus, unsaturated polyester resins that do not greatly shrink when curing and have sufficient mechanical strength are desired.

### BRIEF SUMMARY OF THE INVENTION

In accordance with an embodiment of the invention, a modified layered material is provided, which includes a layered inorganic material intercalated with an organic modifier, wherein the organic modifier includes:

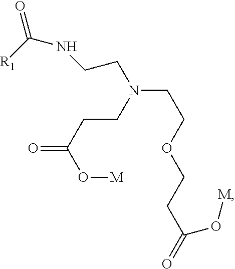

and M includes a metal, and R₁ includes a carbon chain, and X⁻ includes an anion.

In accordance with another embodiment of the invention, an unsaturated polyester nanocomposite is provided, which includes a polymer material including an unsaturated polyester and the modified layered material mentioned above, wherein the modified layered material is dispersed in the polymer material and is at least partially exfoliated.

A detailed description is given in the following embodiments with reference to the accompanying drawings.

### BRIEF DESCRIPTION OF THE DRAWINGS

The present invention can be more fully understood by reading the subsequent detailed description and examples with references made to the accompanying drawings, wherein:

FIG. 1 shows a TEM picture of an unsaturated polyester nanocomposite according to an embodiment of the present invention.

### DETAILED DESCRIPTION OF THE INVENTION

The making and using of the presently preferred embodiments are discussed in detail below. It should be appreciated, however, that the present invention provides many applicable inventive concepts that can be embodied in a wide variety of specific contexts. The specific embodiments discussed are merely illustrative of specific ways to make and use the invention, and do not limit the scope of the invention.

Modified layered materials of embodiments of the present invention are derived by intercalation of an organic modifier into a layered inorganic material. Unsaturated polyester nanocomposites of embodiments of the present invention include a polymer material and the modified layered material, wherein the modified layered material is dispersed in the polymer material and is at least partially exfoliated. The polymer material includes an unsaturated polyester resin. When polymer material and the modified layered material of the embodiment of the invention are combined, the structural stability of the polymer material may be improved.

After crosslinking of a conventional unsaturated polyester resin, the unsaturated polyester resin may shrink significantly, thus negatively affecting the mechanical properties thereof. In embodiments of the present invention, an at least partially exfoliated modified layered material is introduced into an unsaturated polyester resin and dispersed therein. Because of the dispersion of the modified layered material, a three dimensional steric hindrance may be established within the unsaturated polyester resin. Thus, the unsaturated polyester nanocomposite composed of the unsaturated polyester resin and the modified layered material has improved structural stability.

A suitable layered inorganic material according to an embodiment of the invention may be a natural clay or a synthetic layered inorganic material, such as a smectite clay, vermiculite, halloysite, sericite, mica, zirconium phosphate derivatives, layered double hydroxide (LDH), or combinations thereof. Wherein, one kind of smectite clays may include, for example, a montmorillonite, saponite, beidellite, nontronite, hectorite, or combinations thereof. In one embodiment, the layered inorganic material is a cation exchange type, such as a cation exchange type layered inorganic material. The layered inorganic material may have a cation exchange capacity ranging from about 50 meq/100 g to 250 meq/100 g. However, another layered inorganic material having a higher or a lower cation exchange capacity may also be used, such as layered inorganic materials having a cation exchange capacity ranging from about 250 meq/100 g to 500 meq/100 g or ranging from about 10 meq/100 g to 50 meq/100 g.

A suitable organic modifier according to an embodiment of the invention may be capable of intercalating into the layered inorganic material through ion exchange. In one embodiment, a suitable organic modifier has the formula shown as below:

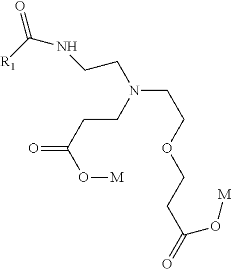

Wherein, M comprises a metal, such as an alkali metal including lithium, sodium, or potassium, and R₁ comprises a carbon chain, such as a long carbon chain. A suitable long carbon chain includes a saturated long carbon chain or an unsaturated long carbon chain. For example, the long carbon chain may have but is not limited to 10 to 30 carbon atoms. In addition, the long carbon chain may further include one or more branch chain(s). The branch chain may include a saturated carbon chain, unsaturated carbon chain, and/or other functional groups.

In another embodiment, a suitable organic modifier has the formula shown as below:

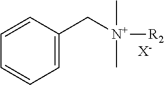

Wherein, X⁻ comprises an anion, such as a halide ion including chloride ion, or iodide ion, and R₂ independent from R₁ comprises a carbon chain, such as a long carbon chain. A suitable long carbon chain includes a saturated long carbon chain or an unsaturated long carbon chain. For example, the long carbon chain may have but is not limited to 10 to 30 carbon atoms. In addition, the long carbon chain may further include one or more branch chain(s). The branch chain may include a saturated carbon chain, an unsaturated carbon chain, and/or other functional groups.

A modified layered material according to an embodiment of the present invention may be prepared by the method described below. First, a layered inorganic material, such as layered clay, is added into deionized water to swell for over 4 hours. The process facilitates an organic modifier to intercalate into the layered inorganic material. Then, a suitable amount of organic modifier may be added. The organic modifier diffuses into the layered inorganic material and bonds thereto. By contacting the layered inorganic material, the organic modifier may bond to the layered inorganic material through ion exchange. In a modified layered material according to an embodiment of the invention, the organic modifier has a weight percentage ranging from about 5 wt % to 30 wt %, based on the total weight of the organic modifier and the layered inorganic material. In another modified layered material according to another embodiment of the invention, the organic modifier has a weight percentage ranging from about 10 wt % to 20 wt %, based on the total weight of the organic modifier and the layered inorganic material.

The interlayer spacing of the layered inorganic material after intercalated with the organic modifier is enlarged, by about 20 Å, due to space occupied by the organic modifier. The modified layered material, with the enlarged interlayer spacing may be mixed with a polymer material to form a modified layered material-polymer composite. Note that it is easier for the modified layered material with an enlarged interlayer spacing to be exfoliated and dispersed in the polymer material.

An unsaturated polyester nanocomposite according to embodiments of the present invention includes a polymer material including the unsaturated polyester and the modified layered material mentioned above. The modified layered material is dispersed in the polymer material and is at least partially exfoliated. In an unsaturated polyester nanocomposite according to an embodiment of the invention, the modified layered material has a weight percentage ranging from about 0.1 wt % to 7 wt %, based on the total weight of the modified layered material and the polymer material. In another unsaturated polyester nanocomposite according to another embodiment of the invention, the modified layered material has a weight percentage ranging from about 1 wt % to 5 wt %, based on the total weight of the modified layered material and the polymer material. In yet another unsaturated polyester nanocomposite according to yet another embodiment of the invention, the modified layered material has a weight percentage of about 3 wt %, based on the total weight of the modified layered material and the polymer material. It should be appreciated that, however, the amount of the modified layered material in the unsaturated polyester nanocomposite of an embodiment of the invention may be adjusted according to intended use, and is not limited to a specific amount.

In one embodiment, the unsaturated polyester nanocomposite may be formed by mixing the modified layered material and the monomer of the polymer material. The polymerization of the monomer of the polymer material may occur between the interlayer space within the modified layered material, for example, by heating or adding catalyst. When the polymerization of the monomer occurs, the modified layered material is exfoliated and dispersed in the formed polymer material. The polymer material, such as an unsaturated polyester resin, may be synthesized by mixing, for example, a variety of unsaturated diacids, a variety of saturated diacids, and a variety of diols to perform a condensation reaction. Suitable unsaturated diacid may include but is not limited to maleic anhydride (MA) and/or fumaric acid (FA). Suitable saturated diacid may include but is not limited to phthalic anhydride (PA), isophthalic acid (IPA), and/or terephthalic acid (TPA). Suitable diol may include but is not limited to propylene glycol (PG), diethylene glycol (DEG), neopentyl glycol (NPG) and/or ethylene glycol (EG).

For example, in one embodiment, the modified layered material is dissolved in a solvent to form a modified layered material solution. Suitable solvent may include but is not limited to a diol solvent, such as propylene glycol. Then, the monomer of the unsaturated polyester resin is added into the modified layered material solution and stirred. Suitable monomer of the unsaturated polyester resin may include but is not limited to a mixture of maleic anhydride and phthalic anhydride. However, it should be appreciated that compounds suitable for forming unsaturated polyester resins are not limited to the examples mentioned above and a variety of other suitable compounds may be adopted to form the unsaturated polyester resins.

After applying a suitable heat treatment to the mixture of the monomer of the unsaturated polyester and the modified layered material, the monomer of the unsaturated polyester may be polymerized to form the polymer material (unsaturated polyester resin) and the modified layered material may be exfoliated and dispersed in the polymer material. For example, the heat treatment may be conducted at about 100° C. to 200° C. for about 15 hours to 20 hours, wherein the temperature may be increased stepwise to carry out the condensation reaction.

In addition, the unsaturated polyester nanocomposite of one embodiment of the invention may be formed by directly blending the modified layered material and the polymer material including the unsaturated polyester resin. For example, a melt blending process or a solution blending process may be applied. Typically, melt blending may be performed to mix the modified layered material and the polymer material in a closed system at a suitable temperature by using, for example, a single screw or double screw extruder, a Banbury mixer, a single screw or double screw extruder mixer, a continuous mixer, or a kneader. The mixing temperature may range from about 150° C. to 250° C. As for solution blending, the modified layered material may be added into a solution containing an organic solvent and the polymer material and stirred at a high speed. After drawing out the solvent, the unsaturated polyester nanocomposite may thus be obtained. For example, an unsaturated polyester resin and a modified layered material may be directly added into a mixer for mixing. The modified layered material is at least partially exfoliated and dispersed in the polymer material including the unsaturated polyester resin due to the strong mechanical force provided by the mixer.

The unsaturated polyester nanocomposite formed according to the methods mentioned above may further be formed into a variety of structures with different shapes or constructions by injection molding, extrude molding, compression molding, or vacuum injection. The unsaturated polyester nanocomposite may also be mixed with other polymer materials or compounds before or after molding.

Because the unsaturated polyester nanocomposite according to an embodiment of the invention includes the modified layered material dispersed and at least partially exfoliated in the polymer material, the structural stability of the polymer material is maintained. Further, shrinkage and the uneven helical patterns that may appear on the surface of the polymer material are reduced, thus effectively enhancing mechanical properties thereof. In addition, the at least partially exfoliated modified layered material may contribute to block out heat thereto, further improving the flame-retardant property of the nanocomposite. The unsaturated polyester nanocomposite according to an embodiment of the invention is suitable for application in ship or bathroom equipment. For example, in one embodiment, the unsaturated polyester nanocomposite may be mixed with glass fibers to form a fiber reinforced plastic (FRP) which may be used as a ship's plate. Due to the introduction of the modified layered material, shrinkage of the polymer material including the unsaturated polyester is significantly reduced, improving the mechanical properties of the products made therefrom. In addition, because the ship's plates or bathroom equipment manufactured using the unsaturated polyester nanocomposite according to an embodiment of the invention have a very flat and smooth surface (minimal uneven helical patterns), the following surface smoothing or polishing process may be prevented or reduced. Thus, the manufacturing cost is reduced significantly and products having better performance may be obtained.

It should be appreciated, however, that the application of the nanocomposite of the present invention are merely exemplary examples and the applications are not limited to specific examples. In addition, the modified layered material of the invention is not limited to be only introduced into a polymer material including an unsaturated polyester resin. In another embodiment, the modified layered material of the invention is introduced into other polymer material systems. In yet another embodiment, the modified layered material of the invention is applied individually in other material systems without being introduced into a polymer material system.

In addition, other additives may be added to the nanocomposite, such as an impact modifier, a hardener, a flame retardant, a compatilizer, an organic or an inorganic filler, an ultraviolet absorber, a light stabilizer, an antistatic agent, a plasticizer, a lubricant, or an antioxidant and so on, according to the intended use.

Some examples are provided as followed for further understanding of the embodiments of the invention.

### Example 1

### Forming a Modified Layered Material

First, montmorillonite clay serving as a layered inorganic material was added into deionized water to swell for over 4 hours. Then, a suitable amount of organic modifier was added to intercalate into the montmorillonite clay through an ion exchange reaction. The organic modifier used in Example 1 had the Formula as shown below:

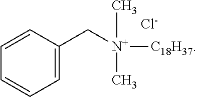

Then, the mixture was repeatedly centrifuged with deionized water and then dried.

X-ray diffraction (XRD) analysis was conducted on the modified layered material obtained in Example 1. When comparing the XRD patterns of the modified layered material obtained in Example 1 and that of a non-modified montmorillonite clay, the results show that the interlayer spacing of the modified layered material was increased from the original 12.61 Å to 23.20 Å. Thus, showing that the organic modifier was intercalated into the montmorillonite clay (layered inorganic material) and enlarged the interlayer spacing of the montmorillonite clay.

### Example 2

### Forming a Modified Layered Material

The modified layered material of Example 2 was prepared by a method similar to that for forming the modified layered material of Example 1. The main difference between Example 1 and Example 2 was that another organic modifier was used. The organic modifier used in Example 2 had the Formula as shown below:

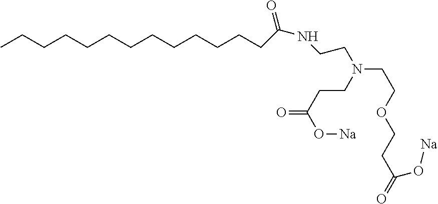

X-ray diffraction (XRD) analysis was conducted on the modified layered material obtained in Example 2. When comparing the XRD patterns of the modified layered material obtained in Example 2 and that of a non-modified montmorillonite clay, the results show that the interlayer spacing of the modified layered material was increased from the original 12.61 Å to 39.20 Å. Thus, showing that the organic modifier was intercalated into the montmorillonite clay (layered inorganic material) and enlarged the interlayer spacing of the montmorillonite clay.

### Example 3

### Forming an Unsaturated Polyester Nanocomposite

197.5 g of propylene glycol and the modified layered material obtained in Example 1 were mixed at a temperature of 80° C. by using a mechanical mixer for over 12 hours.

Then, 92.5 g of maleic anhydride, 210 g of phthalic anhydride, and the premixed propylene glycol/modified layered material solution were put into a reactor vessel and heated. The reaction temperature and reaction time are shown below.

| Temperature | 120~140° C. | →200° C. | 200° C. | Cooling |
|---|---|---|---|---|
| Time | 6 h | 4 h | 6 h | |

After the reaction was finished, the mixture was cooled and diluted. When the temperature was cooled to 80° C., a suitable amount of hydroquinone and styrene were added to dilute the mixture and to control the viscosity of the mixture to be below 300 cps. After stirring for 6 hours, the mixture was removed from the reactor vessel.

The weight percentage of the modified layered material in the unsaturated polyester nanocomposite of Example 3 was controlled by controlling the added amount of the modified layered material of Example 1. In Example 3, the four specimens respectively having weight percentages of 1 wt %, 3 wt %, 5 wt %, and 7 wt %, based on the total weight of the unsaturated polyester nanocomposite, were prepared by a vacuum injection method. The specimens and a comparative specimen were analyzed and compared and the results are shown and discussed in the following description.

### Example 4

### Forming Unsaturated Polyester Nanocomposite

The unsaturated polyester nanocomposite of Example 4 was prepared by a method similar to that for forming the unsaturated polyester nanocomposite of Example 3. The main difference between Example 3 and Example 4 was that the modified layered material of Example 2 was used instead of using the modified layered material of Example 1. In the unsaturated polyester nanocomposite of Example 4, the modified layered material had a weight percentage of 3 wt %, based on the total weight of the unsaturated polyester nanocomposite.

In addition, the unsaturated polyester nanocomposite according to embodiments of the invention can further be mixed with stacked layers of glass fibers and hardened to form a fiber reinforced plastic (FRP) plate. A glass plate serving as a mold was provided and stacked layers of glass fibers were disposed thereon. Then, the unsaturated polyester nanocomposites obtained in Examples 3 and 4 were injected into the mold (stacked layers of glass fibers) by a vacuum injection method. The unsaturated polyester nanocomposites and the stacked layers of glass fibers were hardened together to form fiber reinforced plastic (FRP) plates.

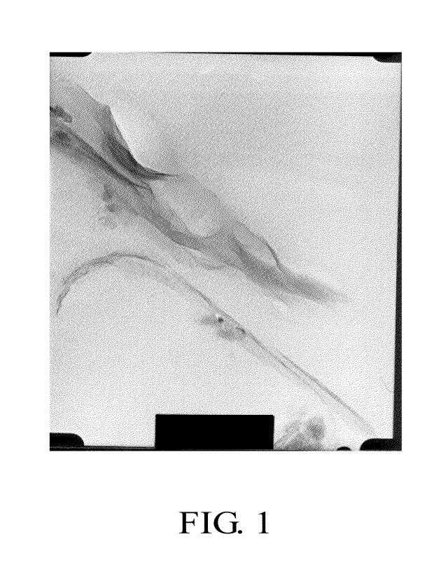

XRD analyses were conducted on the specimens obtained in Examples 3 and 4. The modified layered materials in the polymer material (unsaturated polyester) were shown to have been exfoliated, as the diffraction peaks corresponding to the modified layered material were not present. FIG. 1 shows a TEM picture of an unsaturated polyester nanocomposite with 3 wt % of the modified layered material in Example 3, wherein the magnification of the TEM picture is 60,000 times. As shown in the TEM picture of FIG. 1, the modified layered material did exfoliate and was dispersed in the polymer material.

Table 1 shows the volume shrinkages of the unsaturated polyester nanocomposites of Example 3, the unsaturated polyester nanocomposite of Example 4, and the comparative specimen (without any modified layered material added). The volume shrinkages were determined by an Archimedes method. Table 1 also shows the volume shrinkages of the specimens after being irradiated by UV light source.

TABLE 1

| | Comparative | Example 3 | Example 3 | Example 3 | Example 3 | Example 4 |
|---|---|---|---|---|---|---|
| | specimen | (1 wt %) | (3 wt %) | (5 wt %) | (7 wt %) | (3 wt %) |
| Without | 13.4% | 8.57% | 5.65% | 8.64% | 11.12% | 3.36% |
| irradiated | | | | | | |
| by UV | | | | | | |
| Irradiated | 13.6% | 8.37% | 5.75% | 8.34% | 11.15% | n.a. |
| by UV for | | | | | | |
| 1 hour | | | | | | |
| Irradiated | 13.6% | 8.26% | 5.76% | 8.32% | 11.44% | n.a. |
| by UV for | | | | | | |
| 3 hours | | | | | | |

As shown in Table 1, each example had less volume shrinkage as compared with the comparative specimen. The large shrinkage problem of the unsaturated polyester resins was reduced. In addition, the unsaturated polyester nanocomposite according to an embodiment of the invention was shown to be sunlight-resistant, as volume shrinkage barely changed when the specimens were irradiated by UV light. In the above examples, the unsaturated polyester nanocomposite with 3 wt % of the modified layered material in Example 3. Example 3 (3 wt %), had an especially low volume shrinkage of 5.65%. The volume shrinkage of Example 3 (3 wt %) was reduced significantly by about 7.75% as compared with the comparative specimen without the modified layered material having a volume shrinkage of 13.4%. In addition, the unsaturated polyester nanocomposite with 3 wt % of the modified layered material in Example 4, Example 4 (3 wt %), had an even less volume shrinkage of 3.36%. The volume shrinkage of Example 4 (3 wt %) was significantly reduced by about 10.04% as compared with the comparative specimen having a volume shrinkage of 13.4%. The volume shrinkages of the unsaturated polyester nanocomposite according to the examples mentioned above were reduced by about 2% to 11% (see Table 1) as compared with the comparative specimen without the modified layered material.

FRP Specimen Tests:

Table 2 shows the surface roughness of the FRP specimens formed by the unsaturated polyester nanocomposite of Example 3 and stacked layers of glass fibers. The surface roughness of a comparative FRP specimen without the modified layered material of the invention formed by the same method is also shown in Table 2 for comparison. The manufacturing condition for forming the comparative FRP specimen was controlled to be the same as that for forming the FRP specimens. The surface roughness was determined by using a surface analyzer. Each surface roughness shown in Table 2 is an average of three tested surface roughnesses.

TABLE 2

| | Comparative | Example 3 |
|---|---|---|
| | FRP | (3 wt %) FRP |
| Ra (μm) | 1.38 | 0.69 |
| Rₘₐₓ (μm) | 9.01 | 3.60 |

Ra represents the average roughness, which is a center-line mean roughness. Rmax represents the maximum height difference, which is the height difference between the highest point and the lowest point of the FRP specimen. As shown in Table 2, the FRP specimen prepared by the unsaturated polyester nanocomposite of the invention and stacked layers of glass fibers had a much more even and smooth surface than that of the comparative FRP specimen. Thus, the uneven helical pattern problem that commonly occurs in conventional FRP plates may be reduced. The average roughness of the FRP specimen is reduced by about 0.7 μm as compared with the comparative FRP specimen having no modified layered material added. The maximum height difference of the FRP specimen is reduced by about 5 μm as compared with the comparative FRP specimen having no modified layered material added.

In addition, the FRP specimen including the unsaturated polyester nanocomposite of the invention and the comparative FRP specimen were irradiated by a fluorescent lamp. The reflection image of the fluorescent lamp was observed on the surface of the FRP specimen. When compared with the reflection images on the FRP specimen and the comparative FRP specimen, it was found that the image on the FRP specimen is much clearer than that on the comparative FRP specimen. Thus, showing that the surface of the FRP specimen including the unsaturated polyester nanocomposite of the invention was more even and smooth than that of the comparative FRP specimen.

Table 3 shows the results of a tensile test conducted on the FRP specimens formed by the unsaturated polyester nanocomposite of Example 3 and stacked layers of glass fibers and the comparative FRP specimen. Each of the mechanical properties shown in Table 3 is an average of ten tested values, which were obtained from testing ten specimens with substantially the same sizes.

TABLE 3

| | Comparative | Example 3 |
|---|---|---|
| | FRP | (3 wt %) FRP |
| Maximum load (N) | 5434.128 | 5657.045 |
| Strength (N/mm²) | 291.229 | 325.454 |

As shown in Table 3, the FRP specimen including the unsaturated polyester nanocomposite of the invention sustained larger maximum loads and had higher strength, when compared with the comparative FRP specimen. The strength of the FRP specimen including the unsaturated polyester nanocomposite was increased by about 34N/mm² as compared with the comparative FRP specimen.

Flame-Retardant Property Test:

Table 4 shows the flame-retardant properties of the unsaturated polyester nanocomposites of Example 3 and the comparative specimen (unsaturated polyester resin having no modified layered material added).

TABLE 4

| | Comparative | Example | Example | Example |
|---|---|---|---|---|
| | specimen | 3 (3 wt %) | 3 (5 wt %) | 3 (7 wt %) |
| Average heat | 773.14 | 357.64 | 677.95 | 594.38 |
| release rate | | | | |
| (kW/m²) | | | | |
| Total heat | 136.91 | 42.07 | 139.81 | 132.92 |
| released (MJ/m²) | | | | |

As shown in Table 4, each example had a less than average heat release rate and total heat released as compared with the comparative specimen. Thus, showing significant improvement in the flame-retardant properties of the unsaturated polyester resins. In the above examples, the average heat release rates of the unsaturated polyester nanocomposites of the examples were reduced by about 90 kW/m² to 410 kW/m² as compared with the comparative specimen without the modified layered material (comparative specimen).

In summary, the modified layered material according to an embodiment of the invention was formed through intercalating the organic modifier into the layered inorganic material. The modified layer material of the invention may be introduced into the polymer material including an unsaturated polyester resin. The modified layered material may be exfoliated and dispersed in the polymer material. When compared with an unsaturated polyester resin, the unsaturated polyester nanocomposite according to the embodiment of the invention had significantly reduced volume shrinkage, improved mechanical properties, a more even and smooth surface, and better flame retardant properties. Additionally and particularly, the uneven helical pattern problem of prior art was minimized.

While the invention has been described by way of Example and in terms of the preferred embodiments, it is to be understood that the invention is not limited to the disclosed embodiments. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. A modified layered material, comprising:
a layered inorganic material intercalated with an organic modifier, wherein the organic modifier comprises: 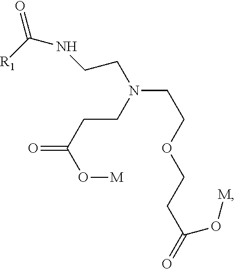
and:
M comprises a metal, and
R₁ comprises a carbon chain.

2. The modified layered material as claimed in claim 1, wherein the organic modifier comprises:

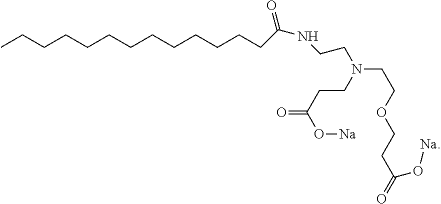

3. The modified layered material as claimed in claim 1, wherein the layered inorganic material comprises a natural clay or synthetic clay.

4. The modified layered material as claimed in claim 1, wherein the layered inorganic material comprises a smectite clay, vermiculite, halloysite, sericite, mica, zirconium phosphate derivatives, layered double hydroxide (LDH), or combinations thereof.

5. The modified layered material as claimed in claim 4, wherein the smectile clay comprises a montmorillonite, saponite, beidellite, nontronite, hectorite, or combinations thereof.

6. The modified layered material as claimed in claim 1, wherein the layered inorganic material has a cation exchange capacity ranging from about 50 meq/100 g to 250 meq/100 g.

7. The modified layered material as claimed in claim 1, wherein the organic modifier has a weight percentage ranging from about 10 wt % to 20 wt %, based on the total weight of the organic modifier and the layered inorganic material.

8. The modified layered material as claimed in claim 1, wherein the modified layered material has an interlayer spacing larger than about 20 Å.

9. An unsaturated polyester nanocomposite, comprising:
a polymer material comprising an unsaturated polyester; and
a modified layered material as claimed in claim 1, wherein the modified layered material is dispersed in the polymer material and is at least partially exfoliated.

10. The unsaturated polyester nanocomposite as claimed in claim 9, wherein the modified layered material has a weight percentage ranging from about 0.1 wt % to 7 wt %, based on the total weight of the modified layered material and the polymer material.

11. The unsaturated polyester nanocomposite as claimed in claim 9, wherein the modified layered material has a weight percentage of about 3 wt %, based on the total weight of the modified layered material and the polymer material.

12. The unsaturated polyester nanocomposite as claimed in claim 9, wherein volume shrinkage of the unsaturated polyester nanocomposite is reduced by about 2% to 11% as compared with the counter part in absence of the modified layered material.

13. The unsaturated polyester nanocomposite as claimed in claim 9, wherein an average heat release rate of the unsaturated polyester nanocomposite is reduced by about 90 kW/m² to 410 kW/m² as compared with the counter part in absence of the modified layered material.
